(19) United States
(12) Patent Application Publication
(10) Pub. No.: US 2025/0209614 A1
(43) Pub. Date: Jun. 26, 2025

(54) AUTOMATIC ANCHOR IMAGE SELECTION

(71) Applicant: Novocure GmbH, Root D4 (CH)

(72) Inventors: Oren PELES ZEEVI, Haifa (IL); Daria HASIN, Haifa (IL)

(73) Assignee: Novecure GmbH, Root D4 (CH)

(21) Appl. No.: 18/958,141

(22) Filed: Nov. 25, 2024

Related U.S. Application Data
(60) Provisional application No. 63/613,817, filed on Dec. 22, 2023.

(51) Int. Cl. G06T 7/00; G06T 17/00; G16H 15/00; G16H 50/50

(52) U.S. Cl. CPC G06T 7/0012; G06T 17/00; G16H 15/00; G16H 50/50; G06T 2207/10081; G06T 2207/10088; G06T 2207/10104; G06T 2207/20092; G06T 2207/30004; G06T 2207/30168; G06T 2210/41

(57) ABSTRACT

A method for reviewing medical images includes: accessing a plurality of medical images of a subject, the medical images comprising at least one of a magnetic resonance imaging (MRI) medical image, a computed tomography (CT) medical image, or a positron emission tomography (PET) medical image, each medical image being a three-dimensional image comprising a plurality of two-dimensional image slices, each medical image comprising voxels; determining values for a plurality of categories for each medical image; determining a score for each medical image based on the values for the categories for each medical image; and identifying one of the medical images as an anchor medical image based on the scores of the medical images, the anchor medical image being used to fix the medical images for creating a three-dimensional model of the subject.

### CROSS-REFERENCE TO RELATED APPLICATIONS

This application claims priority to U.S. Provisional Patent Application No. 63/613,817, filed Dec. 22, 2023, which is incorporated herein by reference in its entirety.

### BACKGROUND

Tumor treating fields (TTFields) are low intensity alternating electric fields within the intermediate frequency range (for example, 50 kHz to 1 MHZ), which may be used to treat tumors as described in U.S. Pat. No. 7,565,205. TTFields are induced non-invasively into a region of interest by transducers placed on the patient's body and applying alternating current (AC) voltages between the transducers. Conventionally, a first pair of transducers and a second pair of transducers are placed on the subject's body. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment.

### BRIEF DESCRIPTION OF THE DRAWINGS

FIGS. 1A and 1B (collectively referred to as FIG. 1) depict an example method for identifying an anchor medical image for creating a three-dimensional model of the subject and further for selecting a transducer layout for applying TTFields to a subject.

FIG. 2 depicts an equation to determine a score for each medical image.

FIGS. 3A and 3B depict a list of medical images with categories of values.

FIGS. 4A-4C depict examples of slices of medical images.

FIG. 5 depicts an example system for delivering TTFields to a subject's body.

FIG. 6 depicts an example placement of transducers on a subject's head.

FIG. 7 depicts an example computer apparatus for use with the embodiments herein.

Various embodiments are described in detail below with reference to the accompanying drawings, wherein like reference numerals represent like elements.

### DESCRIPTION OF EMBODIMENTS

This application describes exemplary methods for selecting a transducer layout for applying TTFields to a subject and apparatuses for applying TTFields based on a transducer layout to the subject's body.

In general, when determining a transducer layout to apply TTFields to a subject, a TTFields planning system requires selection of an anchor medical image among a plurality of medical images of the subject. The anchor medical image may be used to fix the plurality of medical images in order to create a three-dimensional (3D) model of the subject. With a TTFields planning system, the anchor medical image is selected manually, which may take a significant amount of time, especially as each medical image may include several hundred of individual image slices.

The inventors discovered computational techniques to vastly reduce the computation time needed for automatic selection of an anchor medical image. The inventive techniques are particularly integrated into a practical application. With the inventive techniques, automatic selection for an anchor medical image may be performed compared to previous manual techniques. In addition, the automatic selection may be applied to process a higher amount of medical images compared to previous manual techniques. This may result in improved TTFields treatment efficacy in the same computational time.

In particular, the inventors discovered computational techniques that include determining values for a number of categories for each of the medical images and further determining a score for each medical image based on the values for the categories. As such, the anchor medical image may be automatically identified based on the determined scores.

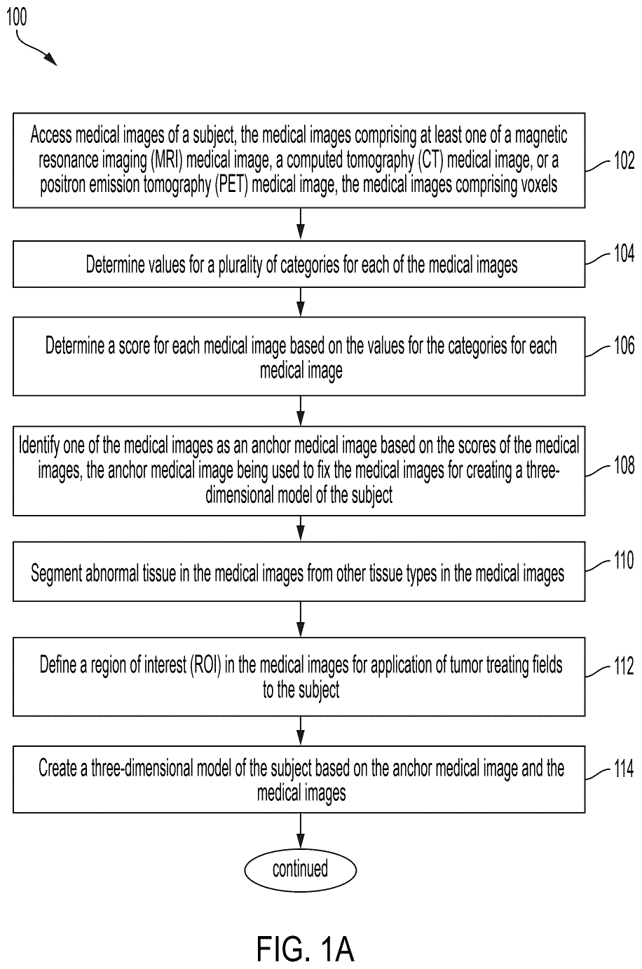

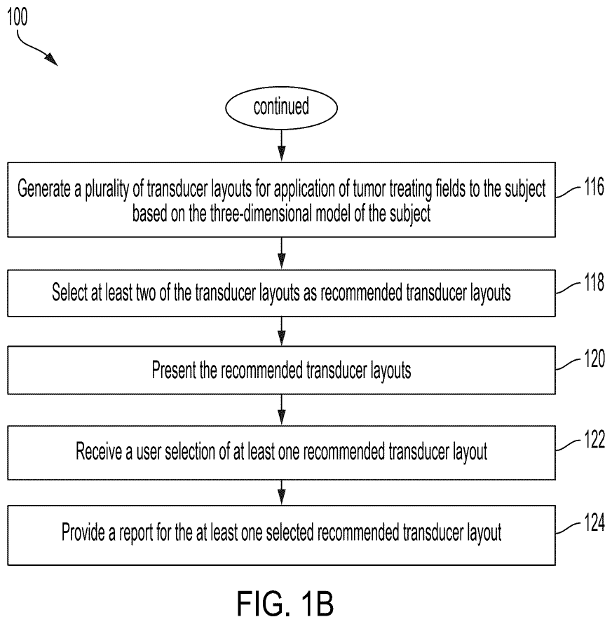

FIGS. 1A and 1B depict an example computer-implemented method 100 for identifying an anchor medical image for creating a 3D model of the subject and further for selecting a transducer layout for applying TTFields to a subject. The method 100 may be implemented by a computer, the computer including one or more processors and memory accessible by the one or more processors, the memory storing instructions that when executed by the one or more processors cause the computer to perform the steps of the method 100. Modifications, additions, or omissions may be made to method 100. While an order of operations is indicated in FIGS. 1A and 1B for illustrative purposes, the timing and ordering of such operations may vary where appropriate without negating the purpose and advantages of the examples set forth in detail herein.

The method 100 may include, at step 102, accessing medical images of the subject. In some embodiments, the medical images may be accessed from memory. As an example, the medical images may be stored locally or accessed from memory over a network. In some embodiments, the medical images may include at least one of a magnetic resonance imaging (MRI) medical image, a computed tomography (CT) medical image, or a positron emission tomography (PET) medical image. In some embodiments, the medical images may include at least one of a plurality of MRI medical images or a plurality of CT medical images. In some embodiments, each medical image may be a 3D image including a plurality of two-dimensional (2D) image slices, and each medical image may include voxels. In some embodiments, the medical images may represent different tissue types of the subject and at least one voxel may be associated with each tissue type. For example, the different tissue types may include organ, muscle, bone, fluid, non-tumor, tumor, etc.

At step 104, the method 100 may include determining values for a plurality of categories for each of the medical images. In some embodiments, the categories of the values may include at least one of: number of slices, slice thickness, reconstruction kernel, reconstruction kernel value (e.g., a numerical value assigned to the reconstruction kernel used based on a perceived quality and/or usefulness of the reconstruction kernel), reconstruction diameter, contrast, contrast value, field of view, time since medical image obtained (e.g., months, days, hours, etc.), x-axis resolution, y-axis resolution, z-axis resolution, x-axis length, y-axis length, z-axis length, front margin of the x-axis length, back margin of the x-axis length, left margin of the y-axis length, right margin of the y-axis length, top margin of the z-axis length, and bottom margin of the z-axis length. In particular, the x-axis may be from front to back of the subject, the y-axis may be from left to right of the subject, and the z-axis may be from top to bottom of the subject. In some embodiments, the categories of the values may preferably include at least six of the above-referenced categories.

In some embodiments, the slice thickness may refer to a thickness of each slice along an axis of how the slices are divided. In some embodiments, the slice thickness may refer to a distance between slices.

The reconstruction kernel may be used in image reconstruction. The reconstruction kernel refer to a filtering process used to modify frequency contents of projection data prior to back projection during image reconstruction in a CT scanner. Different kernels may affect the resulting medical image in different ways (e.g., sharpening certain structures or areas, or blurring certain structures or areas).

The reconstruction diameter may be used in image reconstruction. The reconstruction diameter may refer to a region from which data is used in creating a reconstruction of the medical image.

The contrast may refer to a substance taken by mouth or injected into an intravenous line that causes a particular organ or tissue under study to be seen more clearly.

The contrast value may refer to a binary value that indicates whether or not a contrast agent was used to obtain a medical image.

The field of view may refer to visible dimensions of an exact anatomic region included in a scan. In some embodiments, a full field of view of a medical image may include a full two dimensional image of a subject's body, and a partial field of view may have one or more sections of the subject's body missing from the image. In some embodiments, a full field of view may be critical when planning TTFields treatment, which may require information on an entire portion of a subject's body so as to appropriately determine propagation of electric fields through a subject's body from administering TTFields treatment.

The resolution for an axis may refer to a size of a voxel in a distance measurement, e.g., measured in mm. In some embodiments, the z-axis resolution may indicate a distance between slices of the medical image. As an example, the x-axis resolution and the y-axis resolution may vary between 0.5 mm to 1.2 mm, or between 0.7 mm to 1.0 mm. As an example, the z-axis resolution may vary between 1 mm to 5 mm, or between 0.5 mm to 10 mm.

The length for an axis may indicate a size of the medial image in, for example, voxels or distance. If a medical image is too short, the axis length parameter may be used to eliminate the medial image as an anchor image. As an example, if the medical image has a z-axis length of 20 mm, the medical image may be too short to be an anchor image. As an example, a z-axis length may be at least 50 mm to at least 100 mm. In some embodiments, an axis length may help to indicate how far above or below a region of interest (ROI) a scan was obtained. In some embodiments, an axis length may be critical when planning TTFields treatment, which may require information on an entire portion of a subject's body so as to appropriately determine propagation of electric fields through a subject's body from administering TTFields treatment.

The margin for an axis may refer to a measurement of voxels or distance in reference to (e.g., above, below, or outside) an anatomical landmark. As an example, a top margin of the z-axis length may refer to how far above the lungs an image was obtained, and a bottom margin of the z-axis length may refer to how far below the lungs an image was obtained. In some embodiments, a margin for an axis length may be critical when planning TTFields treatment, which may require information on an entire portion of a subject's body so as to appropriately determine propagation of electric fields through a subject's body from administering TTFields treatment.

As one example, the categories of the values may include the contrast, the field of view, and the z-axis length. As another example, the categories of the values may include the contrast, the z-axis resolution, the field of view, the slice thickness, the reconstruction kernel, and the z-axis length. As another example, the categories of the values may include at least one of the x-axis resolution, the y-axis resolution, or the z-axis resolution. As another example, the categories of the values may include at least one of the x-axis length, the y-axis length, or the z-axis length.

In some embodiments, determining the values may include accessing data stored for each medical image to determine at least one of a resolution along an axis, a number of slices, a reconstruction kernel, or a reconstruction diameter for each medical image, where the axis may be from front to back of the subject, from left to right of the subject, or from top to bottom of the subject. In some embodiments, determining the values may include accessing data stored for each medical image to determine metadata for each medical image, where the data stored for each medical image may be data stored with the medical image or data stored as part of the medical image. In some embodiments, determining the values may include accessing data stored for each medical image to determine a reconstruction kernel for each medical image, and assigning a reconstruction kernel value for each medical image based on the reconstruction kernel for each medical image.

In some embodiments, at least some of the values may be determined by the computer analyzing the medical images. As one example, the values may be determined by analyzing each medical image to assign a binary value as a contrast value for each medical image. For example, a first binary value may be assigned if a the medical image has a contrast value, and a second binary value may be assigned if a the medical image does not have a contrast value. As another example, the values may be determined by analyzing each medical image with a trained machine learning module to determine a contrast value for each medical image. In some other embodiments, the values may be determined by accessing data stored for each medical image to determine a z-axis length for each medical image, wherein a z-axis is from top to bottom of the subject, and analyzing each medical image to determine at least one of a top margin of the z-axis length above a designated area of the subject or a bottom margin of the z-axis length below the designated area of the subject. In some embodiments, analyzing the medical images may be performed by the computer.

At step 106, the method 100 may include determining a score for each medical image based on the values for the plurality of categories for each medical image. In some embodiments, the score for each medical image may be based on a weighted sum of values corresponding to the values for the plurality of categories for each medical image. More specifically, a weight may be assigned for each category and may be further used to determine the weighted sum of values. In some embodiments, the weight for each category may be predetermined. In some embodiments, the weight for each category may be user adjustable.

As one example, the score for each medical image may be based on a weighted sum of at least a contrast value, a normalized field of view value, and a normalized z-axis length value for each medical image, wherein a z-axis is from top to bottom of the subject.

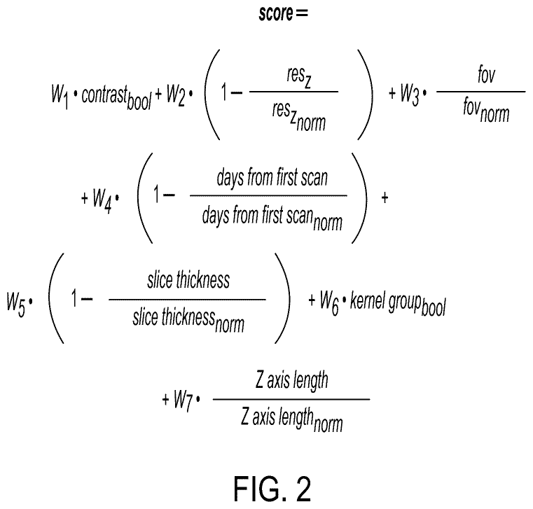

FIG. 2 depicts an equation to determine a score for each medical image. In particular, FIG. 2 illustrates an example where the score for each medical image may be determined based on a weighted sum of values for different categories, namely a contrast value, a normalized z-axis resolution value, a normalized field of view value, a normalized time since medical image obtained (e.g., days since scan of the subject), a normalized slice thickness value, a reconstruction kernel value, and a normalized z-axis length value for each medical image, wherein a z-axis is from top to bottom of the subject. As shown in FIG. 2, weights w₁ to w₇ are assigned to each of these categories, respectively. In particular, weights for each category may or may not be the same. The weights for each category may emphasize importance of one category over another. As one example, the categories for each medical image may include contrast value and field of view, where a weight for a contrast value is larger than a weight for a normalized field of view value. As another example, the categories for each medical image may include field of view and z-axis length, where a weight for a normalized field of view value is larger than a weight for a normalized z-axis length value. As another example, the categories for each medical image may include field of view, resolution, and slice thickness, where a normalized field of view value, a resolution value, and a normalized slice thickness value may have a same weight.

At step 108, the method 100 may include identifying one of the medical images as an anchor medical image based on the scores of the medical images, where the anchor medical image is used to fix the medical images for creating a three-dimensional (3D) model of the subject. In some embodiments, identifying the anchor medical image may include selecting the medical image having a highest score as the anchor medical image. In some embodiments, identifying the anchor medical image may involve manual selection. For example, identifying the anchor medical image by the computer may include selecting at least two of the medical images as recommended medical images based on the scores of the medical images, presenting on a display an indication of the recommended medical images, and receiving user input selecting one of the recommended medical images as the anchor medical image.

At step 110, the method 100 may include segmenting abnormal tissue in the medical images from other tissue types in the medical images. The abnormal tissue may be any undesirable type of tissue, such as a tumor, necrotic tissue, a prior surgical area (e.g., a resection cavity), and/or the like including combinations and/or multiples thereof. In some embodiments, segmenting the abnormal tissue may be based on user input identifying abnormal tissue in the medical images. In some embodiments, segmenting the abnormal tissue may be performed partially or entirely by the computer.

At step 112, the method 100 may include defining a region of interest (ROI) in the medical images for application of TTFields to the subject, where the ROI may be included in the 3D model of the subject. The ROI may define where the TTFields are to focus. In some embodiments, the ROI may be defined based on user input or may be defined partially or entirely by the computer.

At step 114, the method 100 may include creating a 3D model of the subject based on the anchor medical image and the plurality of medical images, where the 3D model of the subject may include the ROI. In some embodiments, the 3D model may include a 3D conductivity map depicting electrical conductivity of tissues of the subject.

In some embodiments, creating the 3D model may include performing calculations to determine conductivity of the tissues of the subject based on the anchor medical image, the plurality of medical images, and the tissue types in the medical images. As one example, creating the 3D model may include assigning tissue types and associated conductivities to voxels of the 3D model of the subject. In some embodiments, creating the 3D model of the subject may include automatically segmenting normal tissue in the medical images. In some embodiments, the 3D model may be created upon receiving use input, such as user approval on the 3D conductivity map associated with the 3D model.

At step 116, the method 100 may include generating a plurality of transducer layouts for application of TTFields to the subject based on the 3D model of the subject. The transducer layouts may define one or more locations, relative to the subject, for placing one or more transducers. In some embodiments, the plurality of the transducer layouts may include four locations on the subject to place four respective transducers, such as on a head or torso of the subject. In some embodiments, each of the transducers may include a plurality of electrode elements. The electrode elements may be any suitable type or material. For example, at least one electrode element may include a ceramic dielectric layer, a polymer film, and/or the like including combinations and/or multiples thereof.

At step 118, the method 100 may include selecting at least two of the transducer layouts as recommended transducer layouts. In some embodiments, the selection may be based on user input, such as one or more pre-determined criteria that a transducer layout must satisfy.

At step 120, the method 100 may include presenting the recommended transducer layouts on a display.

At step 122, the method 100 may include receiving a user selection of at least one recommended transducer layout. The user selection may indicate a preferred transducer layout to apply the TTFields in the ROI of the subject. In some embodiments, the user may select the at least one recommended transducer layout based on one or more criteria, such as convenience of applying TTFields at the recommended transducer layouts and/or such as locations of the recommended transducer layouts.

At step 124, the method 100 may include providing a report for the at least one

selected recommended transducer layout, where the report indicates the selection result from step 122.

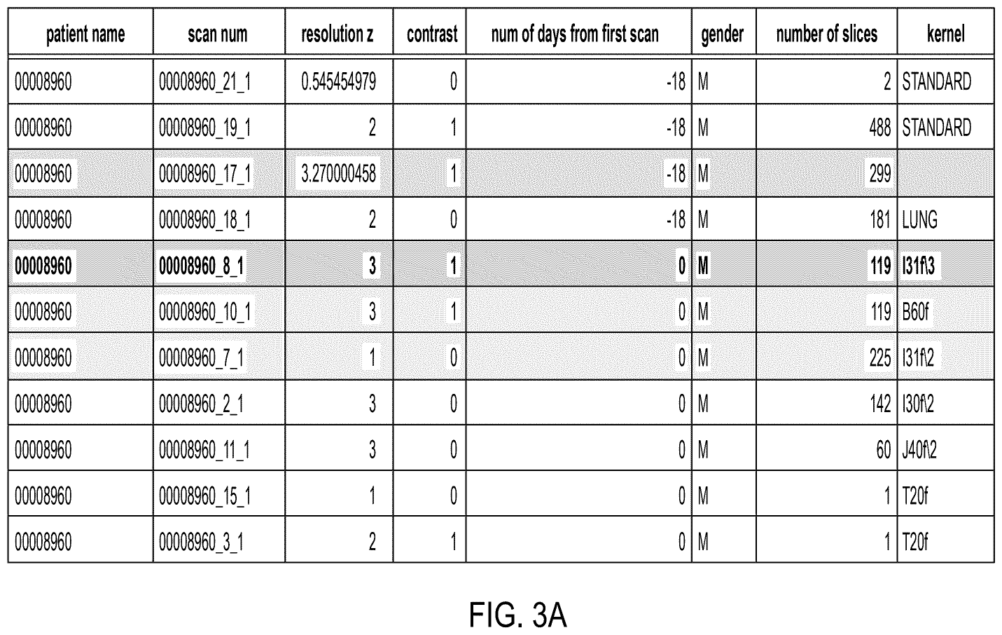

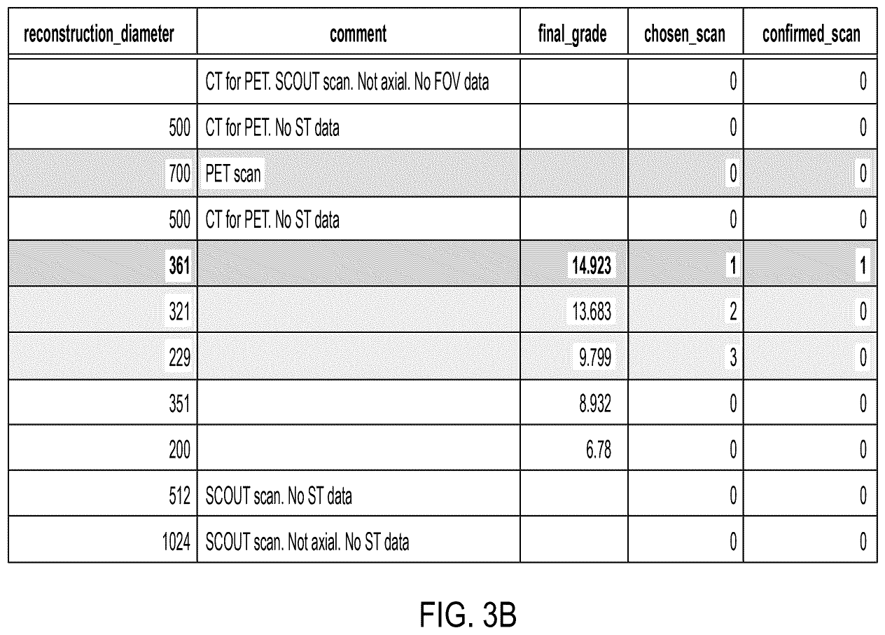

Turning to FIGS. 3A and 3B, FIGS. 3A and 3B depict a list of medical images with categories of values. In particular, FIGS. 3A and 3B illustrate an example of values for a plurality of categories for each medical image, where the categories of the values include z-axis resolution, contrast, time since medical image obtained (e.g., number of days from first scan), number of slices, reconstruction kernel, and reconstruction diameter. In FIG. 3B, the third column from left shows final grade scores generated for each medical image based on the values for the categories for each medical image. The scores are generated based on the equation in FIG. 2. According to FIGS. 3A and 3B, a medical image with scan name of “00008960_8_1” has a highest score of 14.923. A medical image with scan name of “00008960_10_1” has a second highest score of 13.683. A medical image with scan name of “00008960_7_1” has a third highest score of 9.799. As one example, the medical image “00008960_8_1” with the highest score may be automatically selected as the anchor medical image. As another example, these three medical images with the three highest scores may be selected as recommended medical images, and an indication of the recommended medical images may be presented on a display. The anchor medical image may be determined upon receiving user input selecting one of the recommended medical images as the anchor medical image.

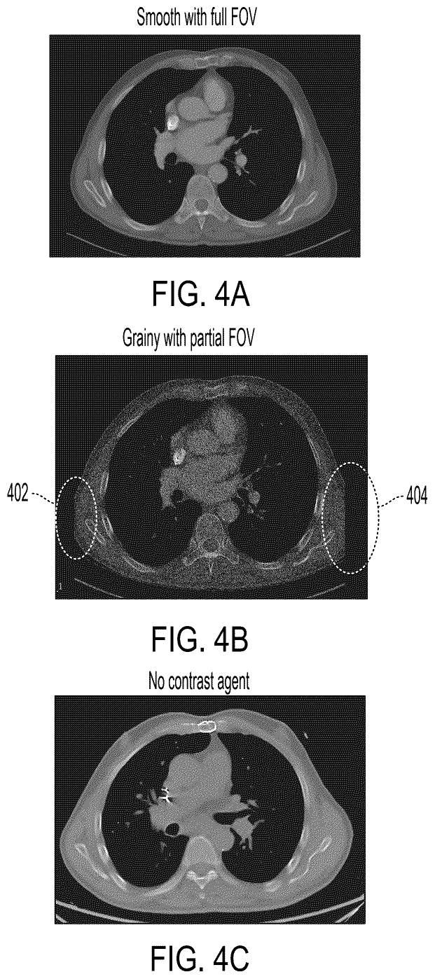

Turning to FIGS. 4A to 4C, FIGS. 4A-4C depict examples of slices of medical images showing an axial view of the lungs of a subject. These medical images are examples of a plurality of medical images of a subject that may be stored as in step 102 and that may be analyzed by a computer as in steps 104 to 108 to designate one of the medical images as an anchor medical image. FIG. 4A depicts an example of a smooth medical image with a complete field of view (FOV) of a subject. In some embodiments, the reconstruction kernel may determine smoothness of the medial image. FIG. 4B depicts an example of a grainy medical image with a partial field of view of a subject, namely the medical image is missing the left and right portions of the subject within the circled sections 402 and 404. FIG. 4C depicts an example of a medical image without a contrast agent. Among the three medical images, FIG. 4A may be selected as the anchor medical image.

### Exemplary Apparatuses

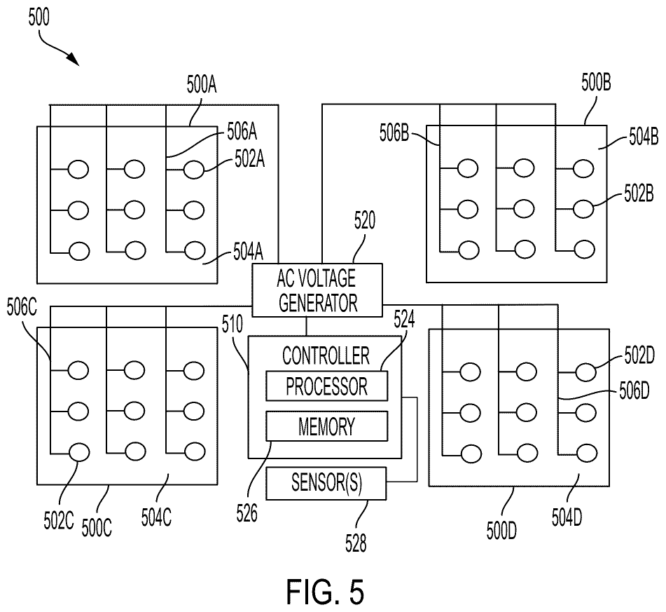

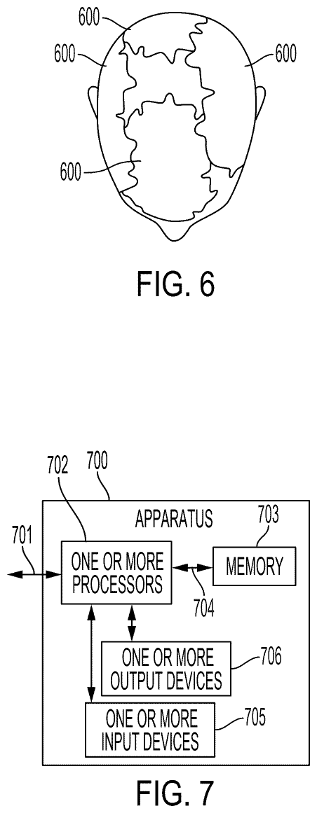

FIG. 5 depicts an example apparatus 500 to apply alternating electric fields (e.g., TTFields) to a subject's body. The system may be used for treating a target region of a subject's body with an alternating electric field (e.g., TTFields). As an example, the target region may be in the subject's brain, and an alternating electric field may be delivered to the subject's body via two pairs of transducers positioned on a head of the subject's body (such as, for example, in FIG. 6, which has four transducers 600). As another example, the target region may be in the subject's torso, and an alternating electric field may be delivered to the subject's body via two pairs of transducers positioned on at least one of a thorax, an abdomen, or one or both thighs of the subject's body. Other transducer placements on the subject's body may be possible.

The example apparatus 500 depicts an example having four transducers 500A-D. Each transducer 500A-D may include substantially flat electrode elements 502A-D positioned on a substrate 504A-D and electrically and physically connected (e.g., through conductive wiring 506A-D). The substrates 504A-D may include, for example, cloth, foam, flexible plastic, and/or conductive medical gel. Two transducers (e.g., 500A and 500D) may be a first pair of transducers configured to apply an alternating electric field to a target region of the subject's body. The other two transducers (e.g., 500B and 500C) may be a second pair of transducers configured to similarly apply an alternating electric field to the target region.

The transducers 500A-D may be coupled to an AC voltage generator 520, and the system may further include a controller 510 communicatively coupled to the AC voltage generator 520. The controller 510 may include a computer having one or more processors 524 and memory 526 accessible by the one or more processors. The memory 526 may store instructions that when executed by the one or more processors control the AC voltage generator 520 to induce alternating electric fields between pairs of the transducers 500A-D according to one or more voltage waveforms and/or cause the computer to perform one or more methods disclosed herein. The controller 510 may monitor operations performed by the AC voltage generator 520 (e.g., via the processor(s) 524). One or more sensor(s) 528 may be coupled to the controller 510 for providing measurement values or other information to the controller 510.

The electrode elements 502A-D may be capacitively coupled. As an example, the electrode elements 502A-D are ceramic electrode elements coupled to each other via conductive wiring 506A-D. When viewed in a direction perpendicular to its face, the ceramic electrode elements may be circular shaped or non-circular shaped. In other embodiments, the array of electrode elements are not capacitively coupled, and there is no dielectric material (such as ceramic, or high dielectric polymer layer) associated with the electrode elements.

The structure of the transducers 500A-D may take many forms. The transducers may be affixed to the subject's body or attached to or incorporated in clothing covering the subject's body. The transducer may include suitable materials for attaching the transducer to the subject's body. For example, the suitable materials may include cloth, foam, flexible plastic, and/or a conductive medical gel. The transducer may be conductive or non-conductive.

The transducer may include any desired number of electrode elements. Various shapes, sizes, and materials may be used for the electrode elements. Any constructions for implementing the transducer (or electric field generating device) for use with embodiments of the invention may be used as long as they are capable of (a) delivering TTFields to the subject's body and (b) being positioned at the locations specified herein. In certain embodiments, at least one electrode element of the first, the second, the third, or the fourth transducer can include at least one ceramic disk that is adapted to generate an alternating electric field. In non-limiting embodiments, at least one electrode element of the first, the second, the third, or the fourth transducer includes a polymer film that is adapted to generate an alternating field.

FIG. 7 depicts an example computer apparatus for use with one or more embodiments described herein. As an example, the apparatus 700 may be a computer to implement certain inventive techniques disclosed herein, such as selecting transducer locations for delivering TTFields to a subject. For example, steps 102 to 124 of FIG. 1 may be performed by a computer, such as the apparatus 700. As an example, the apparatus 700 may be used as the controller 510 of FIG. 5, or as a separate computer apparatus located remote from the controller 510. The apparatus 700 may include one or more processors 702, memory 703, one or more input devices 705, and one or more output devices 706.

Input to the apparatus 700 may be provided by one or more input devices 705, provided from one or more input devices in communication with the apparatus 700 via link 701 (e.g., a wired link or a wireless link; e.g., with a direct connection or over a network), and/or provided from another computer(s) in communication with the apparatus 700 via link 701. As an example, based on input 701, the one or more processors 702 may generate control signals to control the voltage generator. As an example, the input 701 may be user input. As an example, the input 701 may be from another computer in communication with the apparatus 700.

Output for the apparatus 700 may be provided by one or more output devices 706, provided to one or more output devices in communication with the apparatus 700 via link 701, and/or provided from another computer(s) in communication with the apparatus 700 via link 701. The one or more output devices 705 may provide the status of the operation of the invention, such as transducer location selection, voltages being generated, and other operational information. The output device(s) 705 may provide visualization data according to certain embodiments of the invention.

In some embodiments, one or more input devices 705 and one or more output devices 706 may be combined into one or more unitary input/output devices (e.g., a touch screen on a smartphone).

In some embodiments, based on input from one or more input devices 705 or input from outside the apparatus 700 via the link 701, the one or more processors 702 may perform operations as described herein. As an example, user input may be received from the one or more input devices 705. As an example, input may be from another computer in communication with the apparatus 700 via link 701. As an example, input may be from one or more input devices in communication with the apparatus 700 via link 701.

In some embodiments, the one or more processors 702 may perform operations as described herein and provide results of the operations as output. As an example, output may be provided to the one or more output devices 706. As an example, output may be provided to another computer in communication with the apparatus 700 via link 701. As an example, output may be provided to one or more output devices in communication with the apparatus 700 via link 701.

The memory 703 may be accessible by the one or more processors 702 (e.g., via a link 704) so that the one or more processors 702 may read information from and write information to the memory 703. The memory 703 may store instructions that, when executed by the one or more processors 702, implement one or more embodiments described herein. The memory 703 may be a non-transitory computer readable medium (or a non-transitory processor readable medium) containing a set of instructions thereon for reviewing medical images, wherein when executed by a processor (such as one or more processors 702), the instructions cause the processor to perform one or more methods discussed herein.

The apparatus 700 may be an apparatus for reviewing medical images and for selecting transducer locations for delivering tumor treating fields to a subject, the apparatus including: one or more processors (such as one or more processors 702); and memory (such as memory 703) accessible by the one or more processors, the memory storing instructions that when executed by the one or more processors, cause the apparatus to perform one or more methods described herein.

The memory 703 may be a non-transitory processor readable medium containing a set of instructions thereon for reviewing medical images and for selecting transducer locations for delivering tumor treating fields to a subject, wherein when executed by a processor (such as the one or more processors 702), the instructions cause the processor to perform one or more methods described herein.

### Illustrative Embodiments

The invention includes other illustrative embodiments (“Embodiments”) as follows.

Embodiment 1. A computer-implemented method for reviewing medical images, the method comprising: accessing a plurality of medical images of a subject, the medical images comprising at least one of a magnetic resonance imaging (MRI) medical image, a computed tomography (CT) medical image, or a positron emission tomography (PET) medical image, each medical image being a three-dimensional image comprising a plurality of two-dimensional image slices, each medical image comprising voxels; determining values for a plurality of categories for each medical image; determining a score for each medical image based on the values for the categories for each medical image; and identifying one of the medical images as an anchor medical image based on the scores of the medical images, the anchor medical image being used to fix the medical images for creating a three-dimensional model of the subject.

Embodiment 2: The method of Embodiment 1, wherein determining the values comprises: accessing data stored for each medical image to determine at least one of a resolution along an axis, a number of slices, a reconstruction kernel, or a reconstruction diameter for each medical image, wherein the axis is from front to back of the subject, from left to right of the subject, or from top to bottom of the subject.

Embodiment 2A: The method of Embodiment 1, wherein determining the values comprises: accessing data stored for each medical image to determine metadata for each medical image, wherein the data stored for each medical image is data stored with the medical image or data stored as part of the medical image.

Embodiment 3: The method of Embodiment 1, wherein at least some of the values are determined by analyzing the medical images.

Embodiment 4: The method of Embodiment 1, wherein determining the values comprises: analyzing each medical image to assign a binary value as a contrast value for each medical image.

Embodiment 4A: The method of Embodiment 1, wherein determining the values comprises: analyzing each medical image with a trained machine learning module to determine a contrast value for each medical image.

Embodiment 5: The method of Embodiment 1, wherein determining the values comprises: accessing data stored for each medical image to determine a reconstruction kernel for each medical image, and assigning a reconstruction kernel value for each medical image based on the reconstruction kernel for each medical image.

Embodiment 6: The method of Embodiment 1, wherein determining the values comprises: accessing data stored for each medical image to determine a z-axis length for each medical image, wherein a z-axis is from top to bottom of the subject; and analyzing each medical image to determine at least one of a top margin of the z-axis length above a designated area of the subject or a bottom margin of the z-axis length below the designated area of the subject.

Embodiment 7: The method of Embodiment 1, wherein the categories of the values comprise contrast, field of view, and z-axis length, wherein a z-axis is from top to bottom of the subject.

Embodiment 8: The method of Embodiment 1, wherein the categories of the values comprise contrast, z-axis resolution, field of view, slice thickness, reconstruction kernel, and z-axis length, wherein a z-axis is from top to bottom of the subject.

Embodiment 8A: The method of Embodiment 1, wherein the categories of the values comprise at least one of x-axis resolution, y-axis resolution, or z-axis resolution, wherein an x-axis is from front to back of the subject, a y-axis is from left to right of the subject, and a z-axis is from top to bottom of the subject.

Embodiment 8B: The method of Embodiment 1, wherein the categories of the values comprise at least one of x-axis length, y-axis length, or z-axis length, wherein an x-axis is from front to back of the subject, a y-axis is from left to right of the subject, and a z-axis is from top to bottom of the subject.

Embodiment 9: The method of Embodiment 1, wherein the categories of the values comprise at least six of: number of slices, slice thickness, reconstruction kernel, reconstruction kernel value, reconstruction diameter, contrast, contrast value, field of view, time since medical image obtained, x-axis resolution, y-axis resolution, z-axis resolution, x-axis length, y-axis length, z-axis length, front margin of the x-axis length, back margin of the x-axis length, left margin of the y-axis length, right margin of the y-axis length, top margin of the z-axis length, and bottom margin of the z-axis length, wherein an x-axis is from front to back of the subject, a y-axis is from left to right of the subject, and a z-axis is from top to bottom of the subject.

Embodiment 10: The method of Embodiment 1, wherein the score for each medical image is based on a weighted sum of values corresponding to the values for the categories for each medical image.

Embodiment 10A: The method of Embodiment 1, wherein a weight for each category is predetermined, and the weight for each category is used to determine the weighted sum of values.

Embodiment 10B: The method of Embodiment 1, wherein a weight for each category is user adjustable, and the weight for each category is used to determine the weighted sum of values.

Embodiment 11: The method of Embodiment 1, wherein the score for each medical image is based on a weighted sum of at least a contrast value, a normalized field of view value, and a normalized z-axis length value for each medical image, wherein a z-axis is from top to bottom of the subject.

Embodiment 12: The method of Embodiment 1, wherein the score for each medical image is based on a weighted sum of at least a contrast value, a normalized z-axis resolution value, a normalized field of view value, a normalized slice thickness value, a reconstruction kernel value, and a normalized z-axis length value for each medical image, wherein a z-axis is from top to bottom of the subject.

Embodiment 13: The method of Embodiment 1, wherein the score for each medical image is based on a weighted sum of values corresponding to the values for the categories for each medical image, wherein the categories for each medical image comprise contrast value and field of view, wherein a weight for a contrast value is larger than a weight for a normalized field of view value.

Embodiment 14: The method of Embodiment 1, wherein the score for each medical image is based on a weighted sum of values corresponding to the values for the categories for each medical image, wherein the categories for each medical image comprise field of view and z-axis length, wherein a weight for a normalized field of view value is larger than a weight for a normalized z-axis length value, wherein a z-axis is from top to bottom of the subject.

Embodiment 15: The method of Embodiment 1, wherein the score for each medical image is based on a weighted sum of values corresponding to the values for the categories for each medical image, wherein the categories for each medical image comprise field of view, resolution, and slice thickness, wherein a normalized field of view value, a resolution value, and a normalized slice thickness value have a same weight.

Embodiment 16: The method of Embodiment 1, wherein identifying one of the medical images as the anchor medical image comprises selecting the medical image having a highest score as the anchor medical image.

Embodiment 17: The method of Embodiment 1, wherein identifying one of the medical images as the anchor medical image comprises: selecting at least two of the medical images as recommended medical images based on the scores of the medical images; presenting on a display an indication of the recommended medical images; and receiving user input selecting one of the recommended medical images as the anchor medical image.

Embodiment 17A: The method of Embodiment 1, wherein the medical images represent a plurality of tissue types of the subject, wherein at least one voxel is associated with each tissue type.

Embodiment 17B: The method of Embodiment 1, wherein the plurality of medical images further comprises at least one of a plurality of magnetic resonance imaging (MRI) medical images or a plurality of computed tomography (CT) medical images.

Embodiment 18: The method of Embodiment 1, further comprising: creating a three-dimensional model of the subject based on the anchor medical image and the medical images; generating a plurality of transducer layouts for application of tumor treating fields to the subject based on the three-dimensional model of the subject; selecting at least two of the transducer layouts as recommended transducer layouts; presenting the recommended transducer layouts; receiving a user selection of at least one recommended transducer layout; and providing a report for the at least one selected recommended transducer layout.

Embodiment 18A: The method of Embodiment 18, further comprising: segmenting abnormal tissue in the medical images from other tissue types in the medical images; and defining a region of interest (ROI) in the medical images for application of tumor treating fields to the subject, wherein the three-dimensional model of the subject includes the region of interest.

Embodiment 19: A non-transitory processor readable medium containing a set of instructions thereon for reviewing medical images, wherein when executed by a processor, the instructions cause the processor to perform a method comprising: accessing a plurality of medical images of a subject, the medical images comprising at least one of a magnetic resonance imaging (MRI) medical image, a computed tomography (CT) medical image, or a positron emission tomography (PET) medical image, each medical image being a three-dimensional image comprising a plurality of two-dimensional image slices, each medical image comprising voxels; determining values for a plurality of categories for each medical image; determining a score for each medical image based on the values for the categories for each medical image; and identifying one of the medical images as an anchor medical image based on the scores of the medical images, the anchor medical image being used to fix the medical images for creating a three-dimensional model of the subject.

Embodiment 20: An apparatus for reviewing medical images, the apparatus comprising: one or more processors; and memory accessible by the one or more processors, the memory storing instructions that when executed by the one or more processors, cause the apparatus to perform a method comprising: accessing a plurality of medical images of a subject, the medical images comprising at least one of a magnetic resonance imaging (MRI) medical image, a computed tomography (CT) medical image, or a positron emission tomography (PET) medical image, each medical image being a three-dimensional image comprising a plurality of two-dimensional image slices, each medical image comprising voxels; determining values for a plurality of categories for each medical image; determining a score for each medical image based on the values for the categories for each medical image; and identifying one of the medical images as an anchor medical image based on the scores of the medical images, the anchor medical image being used to fix the medical images for creating a three-dimensional model of the subject.

Embodiment 21: A method, machine, manufacture, and/or system substantially as shown and described.

Optionally, for each embodiment described herein, the voltage generation components supply the transducers with an electrical signal having an AC waveform at frequencies in a range from about 50 kHz to about 1 MHz and appropriate to deliver TTFields treatment to the subject's body.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention need not be limited to the described embodiments, but that it has the full scope defined by the language of the following claims, and equivalents thereof.

What is claimed is:

1. A computer-implemented method for reviewing medical images, the method comprising:
accessing a plurality of medical images of a subject, the medical images comprising at least one of a magnetic resonance imaging (MRI) medical image, a computed tomography (CT) medical image, or a positron emission tomography (PET) medical image, each medical image being a three-dimensional image comprising a plurality of two-dimensional image slices, each medical image comprising voxels;
determining values for a plurality of categories for each medical image;
determining a score for each medical image based on the values for the categories for each medical image; and
identifying one of the medical images as an anchor medical image based on the scores of the medical images, the anchor medical image being used to fix the medical images for creating a three-dimensional model of the subject.

2. The method of claim 1, wherein determining the values comprises:
accessing data stored for each medical image to determine at least one of a resolution along an axis, a number of slices, a reconstruction kernel, or a reconstruction diameter for each medical image, wherein the axis is from front to back of the subject, from left to right of the subject, or from top to bottom of the subject.

3. The method of claim 1, wherein at least some of the values are determined by analyzing the medical images.

4. The method of claim 1, wherein determining the values comprises:
analyzing each medical image to assign a binary value as a contrast value for each medical image.

5. The method of claim 1, wherein determining the values comprises:
accessing data stored for each medical image to determine a reconstruction kernel for each medical image, and
assigning a reconstruction kernel value for each medical image based on the reconstruction kernel for each medical image.

6. The method of claim 1, wherein determining the values comprises:
accessing data stored for each medical image to determine a z-axis length for each medical image, wherein a z-axis is from top to bottom of the subject; and
analyzing each medical image to determine at least one of a top margin of the z-axis length above a designated area of the subject or a bottom margin of the z-axis length below the designated area of the subject.

7. The method of claim 1, wherein the categories of the values comprise contrast, field of view, and z-axis length, wherein a z-axis is from top to bottom of the subject.

8. The method of claim 1, wherein the categories of the values comprise contrast, z-axis resolution, field of view, slice thickness, reconstruction kernel, and z-axis length, wherein a z-axis is from top to bottom of the subject.

9. The method of claim 1, wherein the categories of the values comprise at least six of:
number of slices,
slice thickness,
reconstruction kernel,
reconstruction kernel value,
reconstruction diameter,
contrast,
contrast value,
field of view,
time since medical image obtained,
x-axis resolution,
y-axis resolution,
z-axis resolution,
x-axis length,
y-axis length,
z-axis length,
front margin of the x-axis length,
back margin of the x-axis length,
left margin of the y-axis length,
right margin of the y-axis length,
top margin of the z-axis length, and
bottom margin of the z-axis length,
wherein an x-axis is from front to back of the subject, a y-axis is from left to right of the subject, and a z-axis is from top to bottom of the subject.

10. The method of claim 1, wherein the score for each medical image is based on a weighted sum of values corresponding to the values for the categories for each medical image.

11. The method of claim 1, wherein the score for each medical image is based on a weighted sum of at least a contrast value, a normalized field of view value, and a normalized z-axis length value for each medical image, wherein a z-axis is from top to bottom of the subject.

12. The method of claim 1, wherein the score for each medical image is based on a weighted sum of at least a contrast value, a normalized z-axis resolution value, a normalized field of view value, a normalized slice thickness value, a reconstruction kernel value, and a normalized z-axis length value for each medical image, wherein a z-axis is from top to bottom of the subject.

13. The method of claim 1, wherein the score for each medical image is based on a weighted sum of values corresponding to the values for the categories for each medical image, wherein the categories for each medical image comprise contrast value and field of view, wherein a weight for a contrast value is larger than a weight for a normalized field of view value.

14. The method of claim 1, wherein the score for each medical image is based on a weighted sum of values corresponding to the values for the categories for each medical image, wherein the categories for each medical image comprise field of view and z-axis length, wherein a weight for a normalized field of view value is larger than a weight for a normalized z-axis length value, wherein a z-axis is from top to bottom of the subject.

15. The method of claim 1, wherein the score for each medical image is based on a weighted sum of values corresponding to the values for the categories for each medical image, wherein the categories for each medical image comprise field of view, resolution, and slice thickness, wherein a normalized field of view value, a resolution value, and a normalized slice thickness value have a same weight.

16. The method of claim 1, wherein identifying one of the medical images as the anchor medical image comprises selecting the medical image having a highest score as the anchor medical image.

17. The method of claim 1, wherein identifying one of the medical images as the anchor medical image comprises:
selecting at least two of the medical images as recommended medical images based on the scores of the medical images;
presenting on a display an indication of the recommended medical images; and
receiving user input selecting one of the recommended medical images as the anchor medical image.

18. The method of claim 1, further comprising:
creating a three-dimensional model of the subject based on the anchor medical image and the medical images;
generating a plurality of transducer layouts for application of tumor treating fields to the subject based on the three-dimensional model of the subject;
selecting at least two of the transducer layouts as recommended transducer layouts;
presenting the recommended transducer layouts;
receiving a user selection of at least one recommended transducer layout; and
providing a report for the at least one selected recommended transducer layout.

19. A non-transitory processor readable medium containing a set of instructions thereon for reviewing medical images, wherein when executed by a processor, the instructions cause the processor to perform a method comprising:
accessing a plurality of medical images of a subject, the medical images comprising at least one of a magnetic resonance imaging (MRI) medical image, a computed tomography (CT) medical image, or a positron emission tomography (PET) medical image, each medical image being a three-dimensional image comprising a plurality of two-dimensional image slices, each medical image comprising voxels;
determining values for a plurality of categories for each medical image;
determining a score for each medical image based on the values for the categories for each medical image; and
identifying one of the medical images as an anchor medical image based on the scores of the medical images, the anchor medical image being used to fix the medical images for creating a three-dimensional model of the subject.

20. An apparatus for reviewing medical images, the apparatus comprising: one or more processors; and memory accessible by the one or more processors, the memory storing instructions that when executed by the one or more processors, cause the apparatus to perform a method comprising:
accessing a plurality of medical images of a subject, the medical images comprising at least one of a magnetic resonance imaging (MRI) medical image, a computed tomography (CT) medical image, or a positron emission tomography (PET) medical image, each medical image being a three-dimensional image comprising a plurality of two-dimensional image slices, each medical image comprising voxels;
determining values for a plurality of categories for each medical image;
determining a score for each medical image based on the values for the categories for each medical image; and
identifying one of the medical images as an anchor medical image based on the scores of the medical images, the anchor medical image being used to fix the medical images for creating a three-dimensional model of the subject.
